(19) United States
(12) Plant Patent Application Publication
(10) Pub. No.: US 2017/0071101 P1
(43) Pub. Date: Mar. 9, 2017

(54) Phlox plant named 'DPHLOX866'

(50) ;

(71) Applicant: Gavriel Danziger, Moshav Mishmar Hashiva (IL)

(72) Inventor: Gavriel Danziger, Moshav Mishmar Hashiva (IL)

(73) Assignee: DANZIGER "DAN" FLOWER FARM, BEIT DAGAN (IL)

(21) Appl. No.: 14/756,436

(22) Filed: Sep. 4, 2015

(51) Int. Cl. A01H 5/00

(52) U.S. Cl. PLT/320

(57) ABSTRACT

A new and distinct cultivar of Phlox plant named ‘DPHLOX866’, characterized by its upright to outwardly spreading and uniformly mounding plant habit; moderately vigorous growth habit; freely branching habit; dense and full appearance; freely flowering habit; long flowering period; white-colored flowers; and relative tolerance to high temperatures.

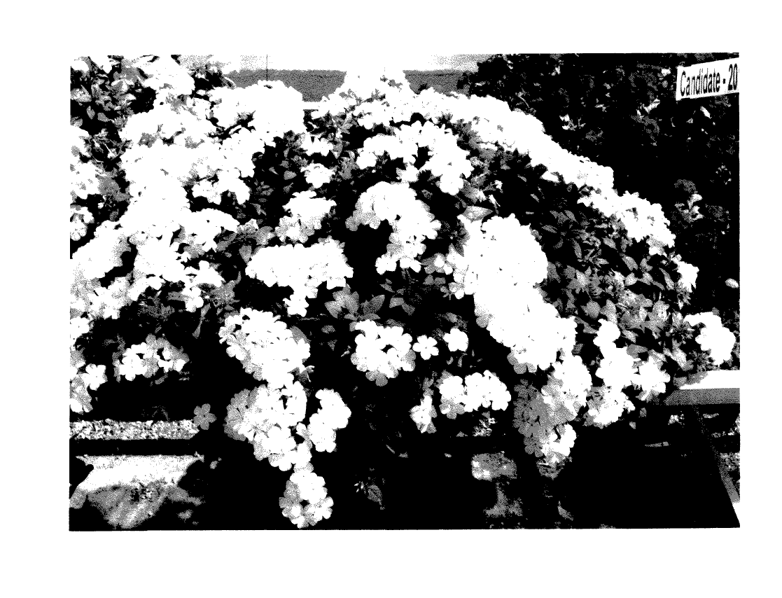

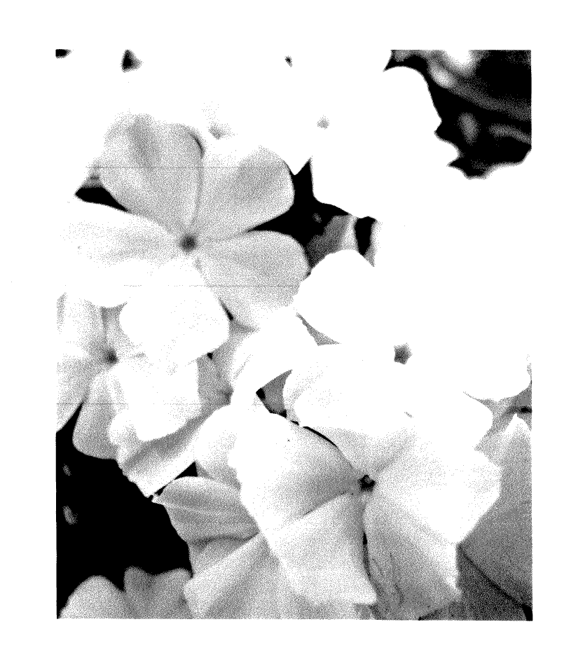

Botanical designation: Phlox drummondii.

Cultivar denomination: ‘DPHLOX866’.

### BACKGROUND OF THE INVENTION

The present invention relates to a new and distinct cultivar of Phlox plant, botanically known as Phlox drummondii and hereinafter referred to by the name ‘DPHLOX866’.

The new Phlox plant is a product of a planned breeding program conducted by the Inventor in Moshav Mishmar Hashiva, Israel. The objective of the breeding program is to create new mounding and full Phlox plants with numerous attractive flowers and low sensitivity to Powdery Mildew.

The new Phlox plant originated from an open-pollination in Moshav Mishmar Hashiva, Israel in October, 2011 of a proprietary selection of Phlox drummondii identified as code number PH-9-1112, not patented, as the female, or seed, parent with an unknown selection of Phlox drummondii as the male, or pollen, parent. The new Phlox plant was discovered and selected by the Inventor as a single flowering plant within the progeny of the stated open-pollination in a controlled greenhouse environment in Moshav Mishmar Hashiva, Israel in April, 2012.

Asexual reproduction of the new Phlox plant by vegetative terminal cuttings in a controlled greenhouse environment in Moshav Mishmar Hashiva, Israel since July, 2012 has shown that the unique features of this new Phlox plant are stable and reproduced true to type in successive generations.

### SUMMARY OF THE INVENTION

Plants of the new Phlox plant have not been observed under all possible combinations of environmental and cultural conditions. The phenotype may vary somewhat with variations in environmental conditions such as temperature and light intensity without, however, any variance in genotype.

The following traits have been repeatedly observed and are determined to be the unique characteristics of ‘DPHLOX866’. These characteristics in combination distinguish ‘DPHLOX866’ as a new and distinct Phlox plant:
1. Upright to outwardly spreading and uniformly mounding plant habit.
2. Moderately vigorous growth habit.
3. Freely branching habit; dense and full appearance.
4. Freely flowering habit; long flowering period.
5. White-colored flowers.
6. Relatively tolerant to high temperatures.

Plants of the new Phlox can be compared to plants of the female parent selection. Plants of the new Phlox differ primarily from plants of the female parent selection in the following characteristics:
1. Plants of the new Phlox are more outwardly spreading than plants of the female parent selection.
2. Plants of the new Phlox are more vigorous than plants of the female parent selection.
3. Plants of the new Phlox and the female parent selection differ in flower color as plants of the female parent selection have purple-colored flowers.

Plants of the new Phlox can be compared to plants of Phlox drummondii ‘Power Phlox White’, not patented. In side-by-side comparisons conducted in Moshav Mishmar Hashiva, Israel, plants of the new Phlox and ‘Power Phlox White’ differed in the following characteristics:
1. Plants of the new Phlox were more mounding than and not as loose and open as plants of ‘Power Phlox White’.
2. Plants of the new Phlox had larger flowers than plants of ‘Power Phlox White’.
3. Plants of the new Phlox and ‘Power Phlox White’ differed in flower color as plants of ‘Power Phlox White’ had white-colored flowers blushed with pink.

### BRIEF DESCRIPTION OF THE PHOTOGRAPHS

The accompanying colored photographs illustrate the overall appearance of the new Phlox plant showing the colors as true as it is reasonably possible to obtain in colored reproductions of this type. Colors in the photographs may differ slightly from the color values cited in the detailed botanical description which accurately describe the colors of the new Phlox plant.

The photograph on the first sheet is a side perspective view of a typical flowering plant of ‘DPHLOX866’ grown in a container.

The photograph on the second sheet is a close-up view of typical flowers of ‘DPHLOX866’.

### DETAILED BOTANICAL DESCRIPTION

The aforementioned photographs and following observations, measurements and values describe plants grown during the autumn in 13-cm containers in an outdoor nursery in Moshav Mishmar Hashiva, Israel and under cultural practices typical of commercial Phlox production. During the production of the plants, day temperatures ranged from 18° C. to 36° C., night temperatures ranged from 10° C. to 17° C. and light levels ranged from 7,000 to 10,000 foot-candles. Plants were pinched one time and were two months old when the photographs and description were taken. Supplemental flower information was collected from plants grown in 22-cm containers during the summer in an outdoor nursery in Loudon, N.H. and under cultural practices typical of commercial Diascia production. During the production of these plants, day temperatures averaged 27° C., night temperatures averaged 13° C. and maximum light levels were 10,000 foot-candles. These plants were pinched one time and were 17 weeks old when the supplemental flower information was taken. In the following description, color references are made to The Royal Horticultural Society Colour Chart, 2007 Edition, except where general terms of ordinary dictionary significance are used.
Botanical classification: Phlox drummondii ‘DPHLOX866’.
Parentage:
   Female, or seed, parent.—Proprietary selection of Phlox drummondii identified as code number PH-9-1112, not patented.
   Male, or pollen, parent.—Unknown selection of Phlox drummondii, not patented.
Propagation:
   Type.—By terminal vegetative cuttings.
   Time to initiate roots, summer.—About five to six days at temperatures about 20° C.
   Time to initiate roots, winter.—About seven to eight days at temperatures ranging from 14° to 18° C.
   Time to produce a rooted young plant, summer.—About three weeks at temperatures ranging from 25° to 35° C.
   Time to produce a rooted young plant, winter.—About four weeks at temperatures ranging from 14° to 20° C.
   Root description.—Medium in thickness, fibrous; typically white in color, actual color of the roots is dependent on substrate composition, water quality, fertilizer type and formulation, substrate temperature and physiological age of roots.
   Rooting habit.—Freely branching; medium density.
Plant description:
   Plant and growth habit.—Herbaceous annual plant; upright to outwardly spreading and uniformly mounding plant habit; broad inverted triangle; moderately vigorous growth habit; freely branching habit with about eleven primary laterals developing per plant, each primary lateral with numerous secondary laterals; dense and bushy appearance.
   Plant height.—About 15 cm.
   Plant width (spread).—About 14 cm.
   Lateral branches.—Length: About 12 cm. Diameter: About 2.5 mm. Internode length: About 2 cm to 3 cm. Strength: Strong. Aspect: About 45° to 80° from vertical. Texture: Pubescent. Color: Close to 146D.
Leaf description:
   Arrangement.—Opposite, simple; sessile.
   Length.—About 4 cm to 5 cm.
   Width.—About 1.5 cm.
   Shape.—Oblong.
   Apex.—Acute.
   Base.—Rounded, clasping.
   Margin.—Entire.
   Texture, upper and lower surfaces.—Pubescent.
   Venation pattern.—Pinnate.
   Color.—Developing and fully expanded leaves, upper surface: Close to 146B; venation, close to 146B. Developing and fully expanded leaves, lower surface: Close to 146C; venation, close to 146C.
Flower description:
   Flower type and flowering habit.—Single rotate salverform flowers arranged in small panicles; flowers face mostly upright to outwardly; freely flowering habit with about four to eight flowers per inflorescence and about 100 flowers per plant.
   Fragrance.—None detected.
   Natural flowering season.—Long flowering season, plants flower continuously from spring until frost in Israel.
   Postproduction longevity.—Flowers last about four days on the plant; flowers not persistent.
   Flower buds.—Height: About 1.2 cm to 1.5 cm. Diameter: About 2.5 mm to 3 mm. Shape: Obovate. Color: Close to 1D.
   Inflorescence height.—About 3.5 cm.
   Inflorescence diameter.—About 5 cm.
   Flower diameter.—About 2.6 cm.
   Flower depth.—About 1.8 cm.
   Flower throat diameter.—About 3 mm.
   Flower tube length: About 1 cm.
   Flower tube diameter, at the base.—About 4 mm.
   Petals.—Quantity per flower and arrangement: Five petals arranged in a single whorl and fused towards the base into a narrow tube. Length from throat: About 1.2 cm. Width: About 1.1 cm. Shape: Obovate. Apex: Rounded, obtuse. Margin: Entire. Texture, petal lobes, upper and lower surfaces: Smooth, glabrous. Texture, throat: Smooth, glabrous. Texture, tube: Tomentose. Luster, petal lobes, upper and lower surfaces: Matte. Luster, throat: Satiny. Luster, tube: Satiny. Color: Developing petals, upper and lower surfaces: Close to 155C. Fully expanded petals, upper and lower surfaces: Close to 155C; color does not change with development. Throat: Close to 155C. Tube: Close to 157A.
   Sepals.—Quantity per flower and arrangement: Five sepals arranged in a single whorl and fused towards the base into a narrow tube. Length: About 8 mm to 10 mm. Width: Less than 1 mm. Shape: Lanceolate. Apex: Acuminate to apiculate. Margin: Entire. Texture, upper and lower surfaces: Smooth, glabrous. Color: When developing, upper and lower surfaces: Distally, close to 146B; proximally, close to 144B. Fully opened, upper and lower surfaces: Distally, close to 146B; proximally, close to 144B.
   Peduncles.—Length: About 1.5 cm to 2 cm. Diameter: About 2 mm. Angle: Upright to about 40° to 45° from vertical. Strength: Strong. Texture: Pubescent. Color: Close to 144B.
   Pedicels.—Length: About 4 mm to 5 mm. Diameter: About 1 mm. Angle: About 30° from peduncle axis. Strength: Strong, flexible. Texture: Pubescent. Color: Close to 144B.
   Reproductive organs.—Stamens: Quantity per flower: Typically five. Anther length: About 1.5 mm. Anther shape: Elliptic to narrowly oblong. Anther color: Close to 12A. Pollen amount: Moderate. Pollen color: Close to 13A. Pistils: Quantity per flower: One. Pistil length: About 4 mm. Stigma shape: Three-parted. Stigma color: Close to 155C. Style length: About 1 mm. Style color: Close to 155C. Ovary color: Close to 144A.
   Fruits.—Length: About 3 mm. Diameter: About 3 mm. Texture: Smooth, glabrous. Color: Close to 144B.
   Seeds.—Quantity per fruit: About two. Length: About 2.2 mm. Diameter: About 1.9 mm. Texture: Smooth, glabrous. Color: Close to 200C.
Disease & pest resistance: Plants of the new Phlox have not been noted to be resistant to pathogens and pests common to Phlox plants.
Temperature tolerance: Plants of the new Phlox have been observed to tolerate temperatures ranging from about 5° C. to about 40° C.

It is claimed:

1. A new and distinct Phlox plant named ‘DPHLOX866’ as illustrated and described.
